(19) United States
(12) Patent Application Publication
(10) Pub. No.: US 2022/0257363 A1
(43) Pub. Date: Aug. 18, 2022

(54) BIFURCATED STENT GRAFTS, STENTS, AND METHODS

(71) Applicant: Endologix LLC, Irvine, CA (US)

(72) Inventors: James Machek, Irvine, CA (US); Christopher Williams, Santa Rosa, CA (US); Christopher Staudenmayer, Irvine, CA (US); Payman Saffari, Irvine, CA (US)

(21) Appl. No.: 17/284,987

(22) PCT Filed: Oct. 23, 2019
(86) PCT No.: PCT/US2019/057652

§ 371 Date: Apr. 13, 2021

Related U.S. Application Data
(60) Provisional application No. 62/750,667, filed on Oct. 25, 2018.

(51) Int. Cl. A61F 2/07; A61F 2/88; A61F 2/06

(52) U.S. Cl. CPC A61F 2/07; A61F 2/88; A61F 2240/001; A61F 2002/065; A61F 2/06

(57) ABSTRACT

A stent includes a first wire and a second wire. The first wire is helically wound along an axis of a main body portion of the stent and along an axis of a first branch portion of the stent. The second wire is helically wound along the axis of the main body portion of the stent and along an axis of a second branch portion of the stent. The first wire and the second wire ar encapsulated in a graft member. A method for making a stent includes winding a first wire helically along a main body portion of a bifurcated mandrel and a first leg portion of the bifurcated mandrel. The method further includes winding a second wire helically along the main body portion of the bifircated mandrel and a second leg portion of the bifurcated mandrel. The first and second wires can be laminated within a graft material.

### CROSS-REFERENCE TO RELATED PATENT APPLICATIONS

This application claims priority from U.S. Provisional Patent App. Ser. No. 62/750,667, filed Oct. 25, 2018, the entire contents of which are incorporated by reference herein.

### FIELD

One or more example embodiments of the present disclosure relate to stents, stent grafts, and methods of manufacturing such stents and stent grafts, and in specific embodiments, to stents, stent grafts, and methods of manufacturing such stents and stent grafts for treating aortoiliac occlusive disease (AIOD).

### BACKGROUND

Aortoiliac occlusive disease (AIOD) is a blockage of the abdominal aorta as it transitions into the common iliac arteries. This blockage is typically caused by a buildup of plaque within the walls of the aorta blood vessels. For example, FIG. 1 is an illustration of a cross section of an example anatomy of an abdominal aorta 10 with aortoiliac occlusive disease centered around an aortic bifurcation 11. In FIG. 1, the aorta 10 branches at the aortic bifurcation 11 into two iliac arteries 12 and 13. Often, plaque 18 is collected and formed on the aortic bifurcation 11 and into the iliac arteries 12 and 13, as well as on an inside wall of the aorta 10 below the renal arteries 15 and 16. As a result of the plaque 18, diameters of the flow lumens in the iliac arteries 12 and 13 are reduced, thereby restricting blood flow to the patient's legs and organs within the pelvis.

Treatment for AIOD generally includes open surgical repair or endoluminal repair. Open surgical repair is often quite successful in patients who are otherwise reasonably healthy and free from significant co-morbidities. Such open surgical procedures are problematic, however, since access to the abdominal aorta is difficult to obtain and because the aorta must be clamped off, placing significant strain on the patient's heart. On the other hand, successful endoluminal procedures have a much shorter recovery period than open surgical procedures.

For endoluminal procedures, two treatment types are generally used for treating AIOD, including kissing stents and covered endovascular reconstruction. Kissing stents is a procedure where two stents are seated at the aortic bifurcation and cross (or kiss) each other above the aortic bifurcation. Similarly, covered endovascular reconstruction is a procedure where a main stent graft body is implanted into the aorta above the aortic bifurcation and separate stent graft branches for each of the iliac arteries are implanted to cross each other within the main stent graft body above the aortic bifurcation. However, both of these treatment types require multiple separate stent grafts that are used to recreate a bifurcation above the diseased aortic bifurcation, and thus, suffer from potential leakage areas where the separate stent grafts are sutured or otherwise pieced together. This is commonly referred to as “radial mismatch,” which can lead to thrombus formation and neointimal hyperplasia. In addition, both of these treatment types create a flow divider that can affect patency. Further, these stents are typically larger than the occlusive vessel blood lumen diameter and are expanded within the vessel, forcing the vessel to stretch and remain patent, which can result in occasional rupture. In the case of rupture of the vessel in combination with the leak channels, patient sequelae can occur. Further, technical success of these procedures can also be difficult as the device placement (or offset of devices) and competition between the stents for endoluminal space can lead to stent occlusion.

Recently, off-label use of a bifurcated stent graft designed for treating abdominal aortic aneurysms (AAA) have been used in experimental treatments for some cases of AIOD. For example, the AFX® Endovascular AAA System from Endologix is a single unit bifurcated stent graft that is designed to treat AAA, but has been used to treat some cases of AIOD. However, the off-label use of a bifurcated AAA implant device can cause difficulties in treating AIOD. For example, aneurysmal stents are built for low radial strength so as not to place excessive force on the diseased tissue. For occlusive disease, high forces are typically desired. Due to this discrepancy, off-label use of AAA devices can result in insufficient radial force, and may require additional ballooning or stent re-enforcement to remain patent. Also, the graft material and the stents of such AAA devices are not attached throughout the length of the device (e.g., they are only attached at the ends), and in many cases, such AAA devices are not sized accordingly (e.g., too long and/or too large in diameter for many patients). Thus, once the AAA device has been placed within smaller anatomies, such as occluded anatomies, it can be difficult to track back through the device without becoming entangled in the stent.

### SUMMARY OF THE DISCLOSURE

A stent in accordance with an embodiment includes a first wire and a second wire. The first wire is helically wound along an axis of a main body portion of the stent and along an axis of a first branch portion of the stent. The second wire is helically wound along the axis of the main body portion of the stent and along an axis of a second branch portion of the stent. In various embodiments, the main body portion of the stent is tubular, the first branch portion of the stent is tubular, and the second branch portion of the stent is tubular. The main body portion of the stent branches to the first branch portion and the second branch portion at a bifurcated portion of the stent.

In various embodiments, windings of the second wire along the main body portion of the stent alternate with windings of the first wire along the main body portion of the stent. In some embodiments, the first wire and the second wire are encapsulated in a graft member along the main body portion, the first wire is encapsulated in the graft member along the first branch portion, and the second wire is encapsulated in the graft member along the second branch portion. Also, in some embodiments, windings of the first wire are only along the main body portion and the first branch portion of the stent, and windings of the second wire are only along the main body portion and the second branch portion of the stent.

In various embodiments, the first wire is an undulating wire, and the second wire is an undulating wire. In some embodiments, an undulation of the first wire has a first side and a second side that meet at a peak, and a length of the first side is shorter than a length of the second side. In some embodiments, an undulation of the first wire has a first side and a second side that meet at a peak, and a length of the first side is equal to a length of the second side.

In various embodiments, the first wire contacts the second wire at two contact areas. In other embodiments, the first wire does not contact the second wire. In some embodiments, the first wire is welded to the second wire at a contact area. In some embodiments, the first wire is crimped to the second wire at a contact area. In various embodiments, a first distance between adjacent windings of the first wire along the main body portion of the stent is greater than a second distance between adjacent windings of the first wire along the first branch portion of the stent.

A stent graft in accordance with an embodiment includes one or more stent members for a main body portion of the stent graft, one or more stent members for a first branch portion of the stent graft, and one or more stent members for a second branch portion of the stent graft. The stent graft further includes a graft member that is a single unit and that holds the one or more stent members for the main body portion of the stent graft, the one or more stent members for the first branch portion of the stent graft, and the one or more stent members for the second branch portion of the stent graft. The graft member is bifurcated at a bifurcated portion of the stent graft to provide the first branch portion and the second branch portion. In various embodiments, the one or more stent members for the main body portion of the stent graft, the one or more stent members for the first branch portion of the stent graft, and the one or more stent members for the second branch portion of the stent graft are laminated within the graft member.

A method in accordance with an embodiment includes winding a first wire of a stent helically along a main body portion of a bifurcated mandrel and a first leg portion of the bifurcated mandrel, and winding a second wire of the stent helically along the main body portion of the bifurcated mandrel and a second leg portion of the bifurcated mandrel. In various embodiments, the method further includes laminating the first wire and the second wire within a graft material. In some embodiments, the first wire is an undulating wire and the second wire is an undulating wire. Also, in some embodiments, an undulation of the first wire has a first side and a second side that meet at a peak, and a length of the first side is shorter than a length of the second side.

### BRIEF DESCRIPTION OF THE DRAWINGS

FIG. 1 is an illustration of a cross section of an example anatomy of an abdominal aorta with aortoiliac occlusive disease centered around an aortic bifurcation.

FIG. 2 shows a stent graft, according to an example embodiment.

FIG. 3A is a perspective view of a stent, according to an example embodiment.

FIG. 3B is an enlarged view of the portion A of the stent shown in FIG. 3A, according to an example embodiment.

FIG. 3C shows a graft member of a stent graft in which the stent of FIG. 3A is laminated in various embodiments.

FIGS. 4A and 4B show a tooling device used in a process of forming a stent of a stent graft, according to an example embodiment.

FIGS. 5A, 5B, 6A, and 6B show various tooling devices having different bifurcation shapes and angles, according to various example embodiments.

FIGS. 7A and 7B show various zig geometries of undulating wires used to form a stent, according to various embodiments.

FIG. 8 is a flow diagram of a method for manufacturing a stent graft, according to an example embodiment.

FIG. 9 shows a stent graft, according to another example embodiment.

FIG. 10 shows a stent graft, according to another example embodiment.

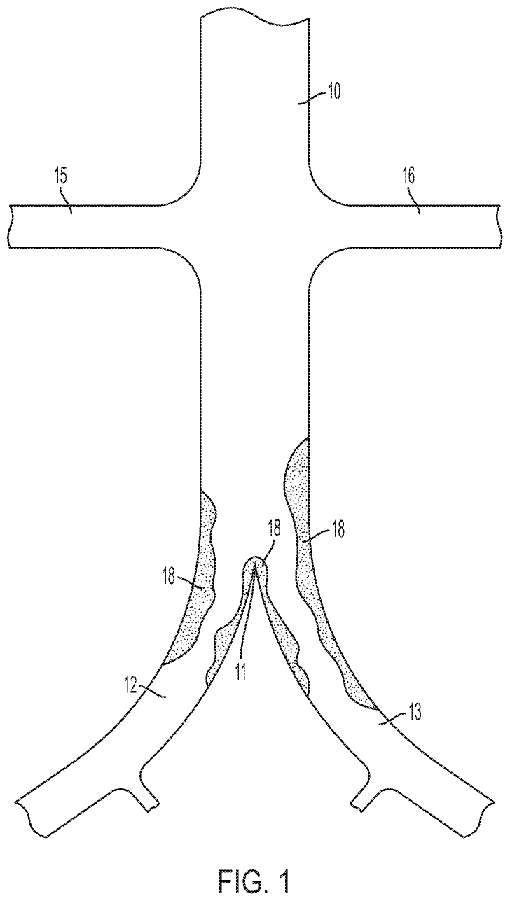

### DETAILED DESCRIPTION

In the following detailed description, reference is made to the accompanying drawings, which form a part of this specification. In the drawings, similar symbols typically identify similar items, unless context dictates otherwise. The illustrative embodiments described in the detailed description and drawings are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure. One aspect described in conjunction with a particular embodiment is not necessarily limited to that embodiment and may be practiced with any other embodiments or arrangements.

One or more aspects of example embodiments are directed to a single unit bifurcated stent graft and a method of manufacturing the same. In various embodiments, a single unit bifurcated stent graft includes a main body, a first branch, and a second branch that is encapsulated or laminated within a graft member so that each of the first and second branches are integrally encapsulated or laminated with the main body. Thus, according to various embodiments, potential leakage areas at a bifurcated portion of the stent graft may be reduced or eliminated when compared to other endoluminal implant systems where a plurality of stent grafts are stitched or otherwise joined together to form the bifurcated portion.

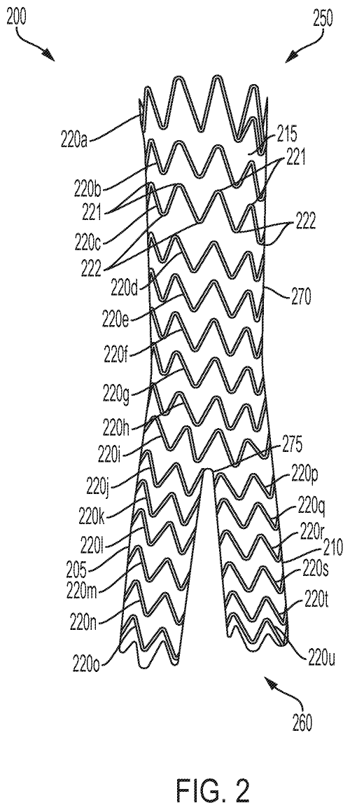

FIG. 2 shows a stent graft 200, according to an example embodiment. In some embodiments, the stent graft 200 is a bifurcated stent graft having a first branch portion 205 (or a first leg) and a second branch portion 210 (or a second leg). The stent graft 200 includes a graft member 215, and stent members 220a, 220b, 220c, 220d, 220e, 220f, 220g, 220h, 220i, 220j, 220k, 220l, 220m, 220n, 220o, 220p, 220q, 220r, 220s, 220t, and 220u. In some embodiments, the stent members 220a, 220c, 220e, 220g, 220i, 220p, 220q, 220r, 220s, 220t, and 220u are connected to each other via a single first stent, and the stent members 220b, 220d, 220f, 220h, 220j, 220k, 220l, 220m, 220n, and 220o are connected to each other via a single second stent. In other embodiments, the stent members 220a, 220b, 220c, 220d, 220e, 220f, 220g, 220h, and 220i are connected to each other via a single first stent, the stent members 220j, 220k, 220l, 220m, 220n, and 220o are connected to each other via a single second stent, and the stent members 220p, 220q, 220r, 220s, 220t, and 220u are connected to each other via a single third stent. In yet other embodiments, each of the stent members 220a, 220b, 220c, 220d, 220e, 220f, 220g, 220h, 220i, 220j, 220k, 220l, 220m, 220n, 220o, 220p, 220q, 220r, 220s, 220t, and 220u are separate from each other (e.g., separate circular rings).

In some embodiments, each of the stent members 220a, 220c, 220e, 220g, 220i, 220p, 220q, 220r, 220s, 220t, and 220u is made of a first wire that is helically wound along an axis in an open tubular configuration, and each of the stent members 220b, 220d, 220f, 220h, 220j, 220k, 220l, 220m, 220n, and 220o is made of a second wire that is helically wound along the axis in the open tubular configuration. In some embodiments, the helically wound wires may be undulating wires having zigs with peaks and valleys. For example, the stent member 220c is depicted as having a plurality of peaks 221 pointing towards a proximal end 250 of the stent graft 200 and a plurality of valleys 222 pointing towards a distal end 260 of the stent graft 200. In various embodiments, each of the stent members 220a, 220b, 220c, 220d, 220e, 220f, 220g, 220h, 220i, 220j, 220k, 220l, 220m, 220n, 220o, 220p, 220q, 220r, 220s, 220t, and 220u forms a crown with a plurality of peaks and valleys.

In various embodiments, each of the wires forming the stent members 220a, 220b, 220c, 220d, 220e, 220f, 220g, 220h, 220i, 220j, 220k, 220l, 220m, 220n, 220o, 220p, 220q, 220r, 220s, 220t, and 220u may be made, for example, from a nickel titanium alloy (NiTi) such as NITINOL, stainless steel, or any other suitable material, including, but not limited to, a cobalt-based alloy such as ELGILOY, platinum, gold, titanium, tantalum, niobium, and/or combinations thereof. In some embodiments, each of the stent members 220a, 220b, 220c, 220d, 220e, 220f, 220g, 220h, 220i, 220j, 220k, 220l, 220m, 220n, 220o, 220p, 220q, 220r, 220s, 220t, and 220u may be balloon-expandable or self-expandable. While the example embodiment in FIG. 2 shows a particular number of stent members, it should be appreciated that, in various embodiments, any suitable number of stent members may be used.

In some embodiments, the stent members 220a, 220b, 220c, 220d, 220e, 220f, 220g, 220h, 220i, 220j, 220k, 220l, 220m, 220n, 220o, 220p, 220q, 220r, 220s, 220t, and 220u are attached to or laminated within the graft member 215. In some embodiments, the graft member 215 extends from the proximal end 250 to the distal end 260 (e.g., ends of the first branch portion 205 and the second branch portion 210). In some other embodiments, the graft member 215 does not cover the entire length of stent graft 200, and may leave the proximal end 250, the distal end 260, or both uncovered, for example. In some embodiments, the stent members 220a, 220b, 220c, 220d, 220e, 220f, 220g, 220h, 220i, 220j, 220k, 220l, 220m, 220n, 220o, 220p, 220q, 220r, 220s, 220t, and 220u are fully laminated or fused within the graft member 215, forming a single unit bifurcated stent graft 200 where the first and second branch portions 205 and 210 are integrally encapsulated with a main body portion 270 of the stent graft 200 by the graft member 215. In this case, the possibility of leakage at a bifurcated portion 275 of the stent graft 200 may be reduced or eliminated, which can occur when a plurality of stent grafts are stitched or otherwise joined together.

In various embodiments, the graft member 215 includes graft material that is made from one or more polymers or other suitable materials. In some embodiments, the graft member 215 is made of polytetrafluoroethylene (PTFE). In some embodiments, the graft member 215 is made of expanded polytetrafluoroethylene (ePTFE). In yet some other embodiments, the stent graft 200 may include at least one additional polymer layer, such as a drug eluting layer, for eluting a bioactive agent from the stent graft 200 after implantation. However, the present disclosure is not limited thereto, and the graft member 215 may include or be made from any suitable graft material.

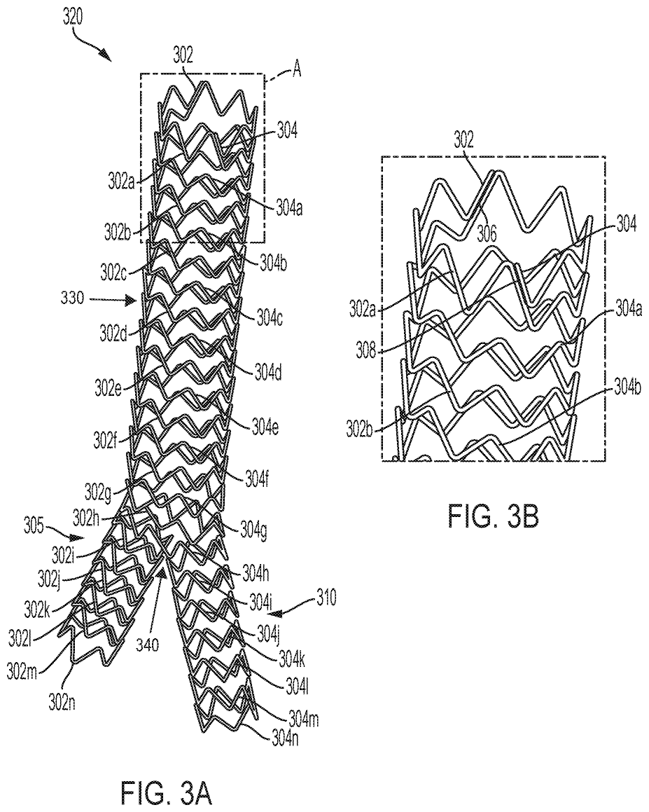

FIG. 3A is a perspective view of a stent, according to an example embodiment, and FIG. 3B is an enlarged view of the portion A of the stent shown in FIG. 3A. Referring to FIGS. 3A and 3B, the stent 320 includes a first wire 302 and a second wire 304. In some embodiments, each of the first and second wires 302 and 304 is an undulating wire. The first wire 302 is helically wound along an axis of the stent 320 in an open tubular configuration and down through a first branch (or leg) portion 305 to form first stent members 302a, 302b, 302c, 302d, 302e, 302f, 302g, 302h, 302i, 302j, 302k, 302l, 302m, and 302n. The first stent members 302i, 302j, 302k, 302l, 302m, and 302n formed by the first wire 302 form the stent of the first branch portion 305. The second wire 304 is helically wound along the axis of the stent 320 in the open tubular configuration and down through a second branch (or leg) portion 310 to form second stent members 304a, 304b, 304c, 304d, 304e, 304f, 304g, 304h, 304i, 304j, 304k, 3041, 304m, and 304n. The second stent members 304i, 304j, 304k, 3041, 304m, and 304n formed by the second wire 304 form the stent of the second branch portion 310.

In some embodiments, the first and second wires 302 and 304 are helically wound along the axis of the stent 320 so that the first stent members 302a, 302b, 302c, 302d, 302e, 302f, 302g, and 302h alternate with the second stent members 304a, 304b, 304c, 304d, 304e, 304f, 304g, and 304h along the axis of the stent 320. On the other hand, the first branch portion 305 includes the first stent members 302i, 302j, 302k, 302l, 302m, and 302n that are wound along an axis of the first branch portion 305, and the second branch portion 310 includes the second stent members 304i, 304j, 304k, 3041, 304m, and 304n that are wound along an axis of the second branch portion 310.

In some embodiments, spacing between adjacent ones of the first stent members 302a, 302b, 302c, 302d, 302e, 302f, 302g, and 302h is greater than spacing between adjacent ones of the first stent members 302i, 302j, 302k, 302l, 302m, and 302n forming the first branch portion 305, so that the second stent members 304a, 304b, 304c, 304d, 304e, 304f, and 304g can be alternately wound between the first stent members 302a, 302b, 302c, 302d, 302e, 302f, 302g, and 302h. Similarly, in some embodiments, spacing between adjacent ones of the second stent members 304a, 304b, 304c, 304d, 304e, 304f, 304g, and 304h is greater than spacing between adjacent ones of the second stent members 304i, 304j, 304k, 3041, 304m, and 304n forming the second branch portion 310, so that the first stent members 302a, 302b, 302c, 302d, 302e, 302f, and 302g can be alternately wound between the second stent members 304a, 304b, 304c, 304d, 304e, 304f, 304g, and 304h.

In some embodiments, the first wire 302 contacts the second wire 304 at only two or less contact points. For example, as shown in FIG. 3B, the first wire 302 contacts the second wire 304 at only a first contact area 306 and a second contact area 308. Other than the two contact areas 306 and 308, in some embodiments the first wire 302 does not contact (or is entirely spaced from) the second wire 304. Similarly, in other embodiments, it should be appreciated that the first wire 302 can contact the second wire 304 at only one contact area. For example, in this case, the first wire 302 can be circularly wound to form the proximal most first stent member (e.g., 302a) and then helically wound thereafter to form the other remaining first stent members. The second wire 304 can contact the first wire 302 at only one contact point at a portion of the proximal most first stent member, and then be helically wound therefrom to form second stent members that alternate with the first stent members. In various embodiments, the first wire 302 is connected to the second wire 304 at the contact area or contact areas (e.g., 306 and 308) by welding, crimping, or the like. In still other examples, the first wire 302 may be entirely spaced from (and does not contact) the second wire 304.

With reference to FIGS. 3A and 3B, the stent 320 in accordance with an embodiment includes the first wire 302 and the second wire 304. The first wire 302 is helically wound along an axis of a main body portion 330 of the stent 320 and along an axis of the first branch portion 305 of the stent 320. The second wire 304 is helically wound along the axis of the main body portion 330 of the stent 320 and along an axis of the second branch portion 310 of the stent 320. In various embodiments, the main body portion 330 of the stent 320 is tubular, the first branch portion 305 of the stent 320 is tubular, and the second branch portion 310 of the stent 320 is tubular. The main body portion 330 of the stent 320 branches to the first branch portion 305 and the second branch portion 310 at a bifurcated portion 340 of the stent 320.

In various embodiments, windings of the second wire 304 along the main body portion 330 of the stent 320 alternate with windings of the first wire 302 along the main body portion 330 of the stent 320. FIG. 3C shows a graft member 350 of a stent graft in accordance with an embodiment. With reference to FIGS. 3A, 3B, and 3C, in some embodiments, the first wire 302 and the second wire 304 are encapsulated in the graft member 350 along the main body portion 330, the first wire 302 is encapsulated in the graft member 350 along the first branch portion 305, and the second wire 304 is encapsulated in the graft member 350 along the second branch portion 310, so as to form a stent graft. Also, in some embodiments, windings of the first wire 302 are only along the main body portion 330 and the first branch portion 305 of the stent 320, and windings of the second wire 304 are only along the main body portion 330 and the second branch portion 310 of the stent 320. In various embodiments, the graft member 350 is a single unit with no stitching.

In various embodiments, the first wire 302 contacts the second wire 304 at two contact areas, such as the first contact area 306 and the second contact area 308. In other embodiments, the first wire 302 does not contact the second wire 304. In some embodiments, the first wire 302 is welded to the second wire 304 at a contact area, such as the first contact area 306. In some embodiments, the first wire 302 is crimped to the second wire 304 at a contact area. In various embodiments, a first distance between adjacent windings of the first wire 302 along the main body portion 330 of the stent 320 is greater than a second distance between adjacent windings of the first wire 302 along the first branch portion 305 of the stent 320.

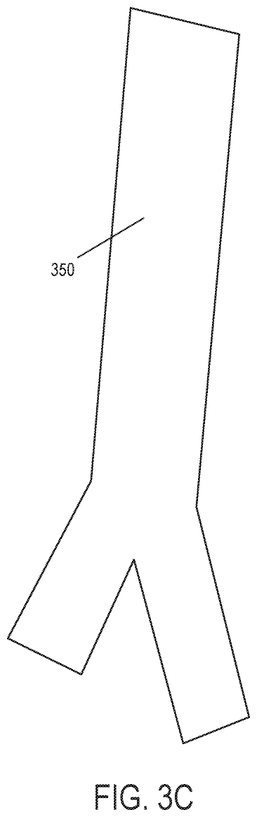

FIGS. 4A and 4B show a tooling device used in a process of forming a stent of a stent graft, according to an example embodiment. First, referring to FIG. 4A, a first undulating wire 402 is helically wound on a bifurcated mandrel 400. The bifurcated mandrel 400 includes a first leg portion 405, a second leg portion 410, and a main body portion 415. In some embodiments, the bifurcated mandrel 400 includes a plurality of pins 420 at a proximal end of the bifurcated mandrel 400 and distal ends of the first and second leg portions 405 and 410 to hold undulating wires at a desired arrangement. The first undulating wire 402 is helically wound along the length of the main body portion 415 and along the first leg portion 405 of the bifurcated mandrel 400. In some embodiments, each of the windings (or first stent members) of the first undulating wire 402 along the main body portion 415 is spaced from adjacent windings (or first stent members) by a first distance d1. In some embodiments, each of the windings (or first stent members) of the first undulating wire 402 along the first leg portion 405 is spaced from adjacent windings (or first stent members) by a second distance d2. In some embodiments, the first distance d1 is greater than the second distance d2, but the present disclosure is not limited thereto, and in other embodiments, d1 can be equal to d2 or even less than d2 depending on rigidity or flexibility considerations of the main stent graft body and/or the branches.

Referring to FIG. 4B, a second undulating wire 404 is helically wound on the bifurcated mandrel 400 shown in FIG. 4A after the first undulating wire 402 has been arranged. As shown in FIG. 4B, the second undulating wire 404 is helically wound along the main body portion 415 of the bifurcated mandrel 400 between the spaces of the first distance d1 of the first undulating wire 402, and helically wound along the second leg portion 410 of the bifurcated mandrel 400. In some embodiments, the windings (or second stent members) of the second undulating wire 404 alternates with the windings (or first stent members) of the first undulating wire 402 along the main body portion 415 of the bifurcated mandrel 400. In some embodiments, each of the windings (or first stent members) of the first undulating wire 402 is spaced from adjacent windings (or second stent members) of the second undulating wire 404 on the main body portion 415 by a third distance d3. In some embodiments, the distance d3 is greater than or equal to the distance d2, but the present disclosure is not limited thereto, and in other embodiments, d3 can be less than d2 depending on rigidity or flexibility considerations of the main stent graft body and/or the branches.

In some embodiments, after the first and second undulating wires 402 and 404 are arranged, the arrangement is baked or otherwise thermally treated to set the arrangement of the first and second undulating wires 402 and 404. In various embodiments, the entire stent including the stent formed on the main body portion 415 by the alternating windings of the first and second undulating wires 402 and 404 and the first and second branches formed by the windings of the first and second undulating wires 402 and 404 on the first and second leg portions 405 and 410, respectively, of the bifurcated mandrel 400 is encapsulated by a graft material, so a single unit bifurcated stent graft is formed (e.g., as shown in FIG. 2). While the example embodiment in FIGS. 4A and 4B shows a particular number of windings (or stent members) of the first and second undulating wires 402 and 404, it should be appreciated that, in various embodiments, any suitable number of windings (or stent members) may be used.

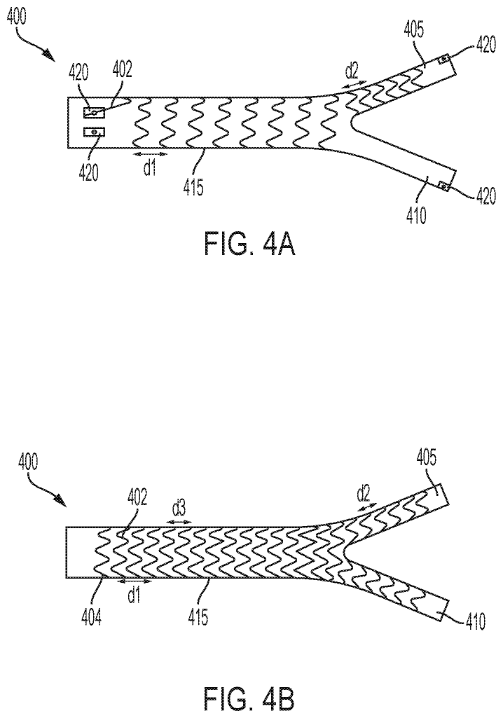

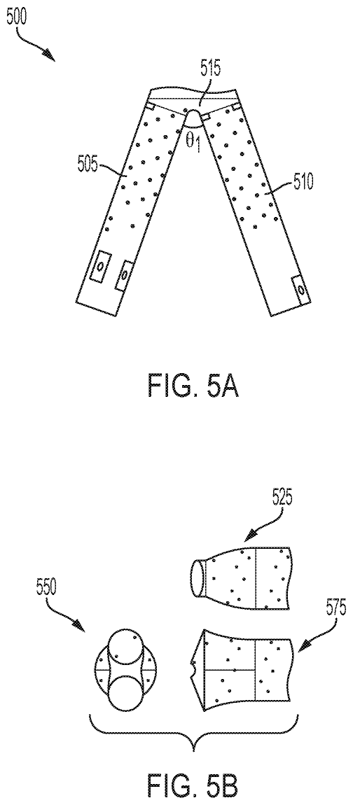

FIGS. 5A, 5B, 6A, and 6B show various tooling devices having different bifurcation shapes and angles, according to various example embodiments. In more detail, FIG. 5A shows a partial front view of a bifurcated portion of a mandrel 500 including a first leg portion 505, a second leg portion 510, and a bifurcation zone 515. Similarly, FIG. 6A shows a partial front view of a bifurcated portion of a mandrel 600 including a first leg portion 605, a second leg portion 610, and a bifurcation zone 615. FIG. 5B shows a side view 525, a bottom view 550, and a back view 575 of the bifurcation zone 515 shown in FIG. 5A with the first and second leg portions 505 and 510 removed. Similarly, FIG. 6B shows a side view 625, a bottom view 650, and a back view 675 of the bifurcation zone 615 shown in FIG. 6A with the first and second leg portions 605 and 610 removed.

Referring to FIGS. 5A and 6A, an angle between leg portions of the mandrel can be variously changed to control the angle between branch portions of a stent graft formed by using the mandrel. For example, the mandrel 500 of FIG. 5A has a first angle θ1 between the first leg portion 505 and the second leg portion 510. The mandrel 600 of FIG. 6A has a second angle θ₂ between the first leg portion 605 and the second leg portion 610. Referring to FIGS. 5A and 6A, the first angle θ1 may be smaller than the second angle θ₂. In this case, a bifurcated stent graft formed using the mandrel 500 will have an angle between first and second branch portions that is smaller than the angle between the first and second branch portions of a bifurcated stent graft formed using the mandrel 600.

Referring to FIGS. 5A, 5B, 6A, and 6B, the shapes and sizes of the leg portions or main body portions of the mandrel can also be variously changed to control the shapes and sizes of the branch portions and the main body portions of a bifurcated stent graft formed using the mandrel. For example, the mandrel 500 can have a more gradually tapered shape between a main body portion and the leg portions 505 and 510 as shown in the side and back views 525 and 575 than the mandrel 600 as shown in the side and back views 625 and 675. Further, the mandrel 600 can have more of a rounded shape for a main body to branch portion transition as shown in the bottom view 650 than the mandrel 500 as shown in the bottom view 550. Accordingly, the shapes and sizes of a bifurcated stent graft formed by using the mandrels 500 and 600 may be variously modified corresponding to the shapes and sizes of the mandrel used to form the bifurcated stent graft.

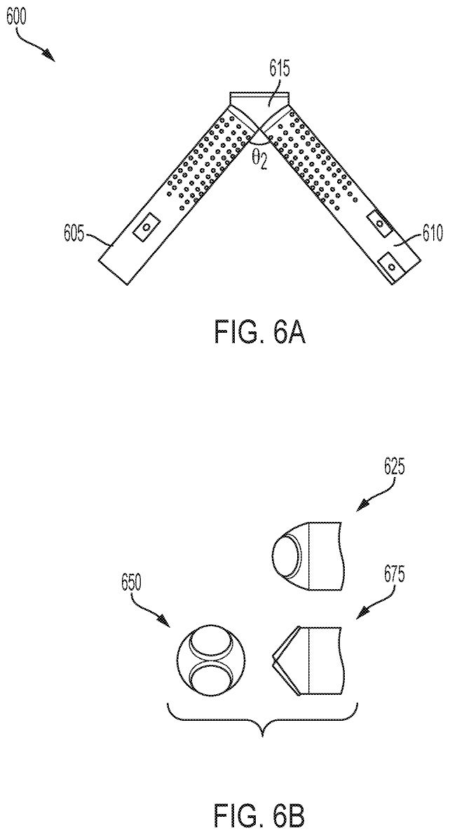

FIGS. 7A and 7B show various zig geometries of the undulating wires used to form a stent, according to various embodiments. In various embodiments, the undulating wires used to form the stent can be helically wound by controlling the lengths of the zigs, and/or by controlling an angle of the windings. For example, as shown in FIG. 7A, one undulation of the undulating wire can have a first side 702 and a second side 704 that define a peak 703. In some embodiments, a length of the first side 702 may be shorter than a length of the second side 704. In this case, when the undulating wire is helically wound, the resulting structure will gradually lengthen in the winding direction. Similarly, in some embodiments, a distance between windings of the undulating wire may be controlled by the lengths of the sides of the undulations.

On the other hand, in some embodiments, as shown in FIG. 7B, one undulation of the undulating wire can have a first side 706 and a second side 708 that define a peak 707. In some embodiments, a length of the first side 706 may be equal to a length of the second side 708. In this case, if the undulating wire is wound in a direction normal to the axis of the main stent graft body, the undulating wire will be circularly wound. On the other hand, if the undulating wire is wound at an angle with respect to the normal direction, the undulating wire will lengthen in the winding direction. Similarly, in some embodiments, a distance between windings of the undulating wire may be controlled by the angle of the windings with respect to the normal direction. Accordingly, in various embodiments, the undulating wire may be helically wound to form the stent by having different lengths of the zigs, by controlling the angle with respect to the normal direction, and/or a combination thereof.

With reference to FIGS. 3A and 7A, in various embodiments, the first wire 302 is an undulating wire, and the second wire 304 is an undulating wire. In some embodiments, an undulation of the first wire 302 has a first side 702 and a second side 704 that meet at a peak 703, and a length of the first side 702 is shorter than a length of the second side 704. With reference to FIGS. 3A and 7B, in some embodiments, an undulation of the first wire 302 has a first side 706 and a second side 708 that meet at a peak 707, and a length of the first side 706 is equal to a length of the second side 708.

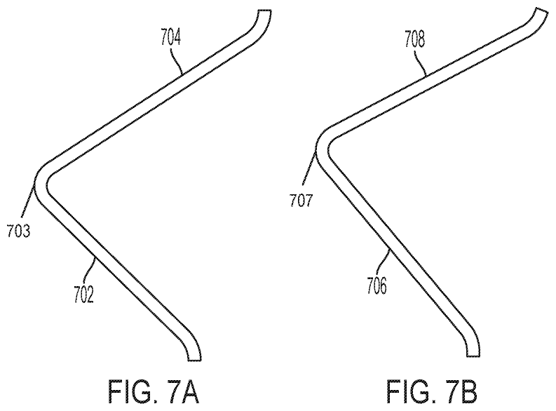

FIG. 8 is a flow diagram of a method for manufacturing a stent graft, according to an example embodiment. Referring to FIG. 8, the method 800 starts and a bifurcated mandrel having a main body portion, a first leg portion, and a second leg portion is provided at block 805. For example, in various embodiments, the bifurcated mandrel may be the same as or similar to any of the mandrels 400, 500, or 600 shown in FIGS. 4A, 5A, or 6A.

With reference to FIG. 8, a first wire is helically wound along a length of the main body portion and along a length of the first leg portion at block 810. In some embodiments, the first wire is an undulating wire having peaks and valleys. In some embodiments, spacing between adjacent windings of the first wire along the main body portion is greater than spacing between adjacent windings of the first wire along the first leg portion.

A second wire is helically wound along the length of the main body portion and along a length of the second leg portion at block 815. In some embodiments, the second wire is an undulating wire having peaks and valleys. In some embodiments, spacing between adjacent windings of the second wire along the main body portion is greater than spacing between adjacent windings of the second wire along the first leg portion. In some embodiments, windings of the second wire along the main body portion is alternately arranged with windings of the first wire along the main body portion. In some embodiments, spacing between adjacent first and second windings is equal to the spacing between the adjacent windings of the first leg portion or the second leg portion. In other embodiments, spacing between the adjacent first and second windings is greater than the spacing between the adjacent windings of the first leg portion or the second leg portion.

In some embodiments, the spacing between windings may be controlled based on an angle of the windings with respect to a direction normal to an axis of the main body portion, the first leg portion, or the second leg portion. In some embodiments, the spacing between the windings may be controlled based on lengths of zigs of the undulating wire. In some embodiments, the spacing between the windings may be controlled based on a combination of the angles and the lengths of the zigs.

The first and second undulating wires including the windings on the main body portion and the windings on the first and second leg portions are laminated or encapsulated within a graft material at block 820. In some embodiments, the graft material extends from a proximal end of the main body portion to distal ends of the first and second leg portions. In some embodiments, all of the windings of the first and second wires on the main body portion and on the first and second leg portions are fully laminated or fused within the graft material. Accordingly, in some embodiments, a single unit bifurcated stent graft is formed where the branches are integrally encapsulated with the main body of the bifurcated stent graft by the graft material.

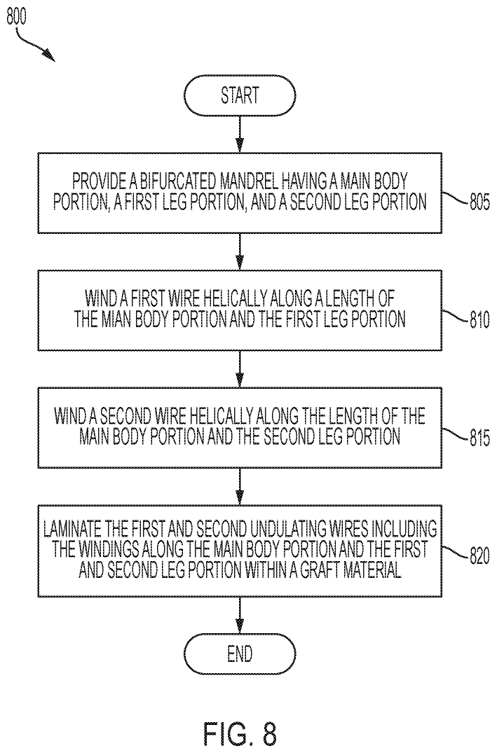

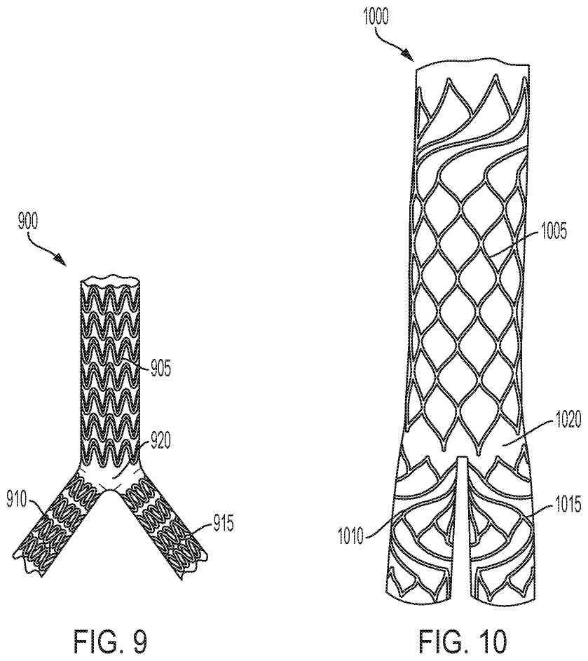

FIGS. 9 and 10 show various examples of a bifurcated stent graft, according to other example embodiments. Referring to FIG. 9, in some embodiments, a bifurcated stent graft 900 includes a first stent 905, a second stent 910, and a third stent 915. The first stent 905 may form a stent for the main stent graft body, the second stent 910 may form a stent for the first branch (or leg), and the third stent 915 may form a stent for the second branch (or leg). In some embodiments, the first stent 905 may be formed by a first undulating wire that is helically wound along an axis of the main stent graft body, the second stent 910 may be formed by a second undulating wire that is helically wound along an axis of the first branch, and the third stent 915 may be formed by a third undulating wire that is helically wound along an axis of the second branch.

In some embodiments, each of the first, second, and third stents 905, 910, and 915 may be laminated, encapsulated, or otherwise attached to a graft member 920. In some embodiments, the graft member 920 extends from a proximal end of the first stent 905 to distal ends of the second and third stents 910 and 915. In some other embodiments, the graft member 920 does not cover the entire length of stent graft 900, and may leave the proximal end, the distal ends, or both uncovered, for example. In some embodiments, each of the first, second, and third stents 905, 910, and 915 is fully laminated or fused within the graft member 920, forming a single unit bifurcated stent graft 900 where the branch portions formed by the second and third stents 910 and 915 are integrally encapsulated with the main body portion formed by the first stent 905 by the graft member 920. In this case, the possibility of leakage at the bifurcated portion of the stent graft 900 may be reduced or eliminated, which can occur when a plurality of stent grafts are stitched or otherwise joined together to form a bifurcated portion.

Referring to FIG. 10, in some embodiments, a bifurcated stent graft 1000 includes a first stent 1005, a second stent 1010, and a third stent 1015. The first stent 1005 may form the stent for a main stent graft body, the second stent 1010 may form the stent for a first branch (or leg), and the third stent 1015 may form the stent for a second branch (or leg). In some embodiments, each of the first, second, and third stents 1005, 1010, and 1015 may be formed from laser cutting of a tubular sheet (e.g., NITINOL tubular sheet). In various embodiments, the first, second, and third stents 1005, 1010, and 1015 may be connected to (or contact) each other or may be spaced apart from each other.

In some embodiments, each of the first, second, and third stents 1005, 1010, and 1015 may be laminated, encapsulated, or otherwise attached to a graft member 1020. In some embodiments, the graft member 1020 extends from a proximal end of the first stent 1005 to distal ends of the second and third stents 1010 and 1015. In some other embodiments, the graft member 1020 does not cover the entire length of stent graft 1000, and may leave the proximal end, the distal ends, or both uncovered, for example. In some embodiments, each of the first, second, and third stents 1005, 1010, and 1015 is fully laminated or fused within the graft member 1020, forming a single unit bifurcated stent graft 1000 where the branch portions formed by the second and third stents 1010 and 1015 are integrally encapsulated with the main body portion formed by the first stent 1005 by the graft member 1020. In this case, the possibility of leakage at the bifurcated portion of the stent graft 1000 may be reduced or eliminated, which can occur when a plurality of stent grafts are stitched or otherwise joined together to form a bifurcated portion.

In the drawings, the relative sizes of elements, layers, and regions may be exaggerated and/or simplified for clarity. Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described above could be termed a second element, component, region, layer or section, without departing from the spirit and scope of the present disclosure.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” and “including,” “has,” “have,” and “having,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

The embodiments disclosed herein are to be considered in all respects as illustrative, and not restrictive of the present disclosure. The present disclosure is in no way limited to the embodiments described above. Various modifications and changes may be made to the embodiments without departing from the spirit and scope of the present disclosure.

What is claimed is:

1. A stent, comprising:
a first wire that is helically wound along an axis of a main body portion of the stent and along an axis of a first branch portion of the stent; and
a second wire that is helically wound along the axis of the main body portion of the stent and along an axis of a second branch portion of the stent.

2. The stent of claim 1,
wherein the main body portion of the stent is tubular;
wherein the first branch portion of the stent is tubular; and
wherein the second branch portion of the stent is tubular.

3. The stent of claim 1,
wherein the main body portion of the stent branches to the first branch portion and the second branch portion at a bifurcated portion of the stent.

4. The stent of claim 1,
wherein windings of the second wire along the main body portion of the stent alternate with windings of the first wire along the main body portion of the stent.

5. The stent of claim 1,
wherein the first wire and the second wire are encapsulated in a graft member along the main body portion;
wherein the first wire is encapsulated in the graft member along the first branch portion; and
wherein the second wire is encapsulated in the graft member along the second branch portion.

6. The stent of claim 1,
wherein windings of the first wire are only along the main body portion and the first branch portion of the stent, and windings of the second wire are only along the main body portion and the second branch portion of the stent.

7. The stent of claim 1,
wherein the first wire is an undulating wire; and
wherein the second wire is an undulating wire.

8. The stent of claim 7,
wherein an undulation of the first wire has a first side and a second side that meet at a peak; and
wherein a length of the first side is shorter than a length of the second side.

9. The stent of claim 7,
wherein an undulation of the first wire has a first side and a second side that meet at a peak; and
wherein a length of the first side is equal to a length of the second side.

10. The stent of claim 1,
wherein the first wire contacts the second wire at two contact areas.

11. The stent of claim 1,
wherein the first wire does not contact the second wire.

12. The stent of claim 1,
wherein the first wire is welded to the second wire at a contact area.

13. The stent of claim 1,
wherein the first wire is crimped to the second wire at a contact area.

14. The stent of claim 1,
wherein a first distance between adjacent windings of the first wire along the main body portion of the stent is greater than a second distance between adjacent windings of the first wire along the first branch portion of the stent.

15. A stent graft, comprising:
one or more stent members for a main body portion of the stent graft;
one or more stent members for a first branch portion of the stent graft;
one or more stent members for a second branch portion of the stent graft; and
a graft member that is a single unit and that holds the one or more stent members for the main body portion of the stent graft, the one or more stent members for the first branch portion of the stent graft, and the one or more stent members for the second branch portion of the stent graft;
wherein the graft member is bifurcated at a bifurcated portion of the stent graft to provide the first branch portion and the second branch portion.

16. The stent graft of claim 15,
wherein the one or more stent members for the main body portion of the stent graft, the one or more stent members for the first branch portion of the stent graft, and the one or more stent members for the second branch portion of the stent graft are laminated within the graft member.

17. A method, comprising:
winding a first wire of a stent helically along a main body portion of a bifurcated mandrel and a first leg portion of the bifurcated mandrel;
winding a second wire of the stent helically along the main body portion of the bifurcated mandrel and a second leg portion of the bifurcated mandrel.

18. The method of claim 17, further comprising:
laminating the first wire and the second wire within a graft material.

19. The method of claim 17,
wherein the first wire is an undulating wire; and
wherein the second wire is an undulating wire.

20. The method of claim 19,
wherein an undulation of the first wire has a first side and a second side that meet at a peak, and
wherein a length of the first side is shorter than a length of the second side.
